(19) United States
(12) Patent Application Publication
(10) Pub. No.: US 2007/0142257 A1
(43) Pub. Date: Jun. 21, 2007

(54) Soap Composition with Encapsulates

(76) Inventor: Regina Hourigan, Metuchen, NJ (US)

Correspondence Address: COLGATE-PALMOLIVE COMPANY, 909 RIVER ROAD, PISCATAWAY, NJ, 08855, US

(21) Appl. No.: 11/457,520

(22) Filed: Jul. 14, 2006

Related U.S. Application Data
(60) Provisional application No. 60/752,345, filed on Dec. 21, 2005.

(51) Int. Cl. A61K 8/02

(52) U.S. Cl. 510/141

(57) ABSTRACT

A personal care composition comprising a product base with at least one encapsulate dispersed throughout is disclosed. The at least one encapsulate contains a hydrophobic internal phase comprising a density-modifying agent such that the specific gravity of the solution is adjusted to provide for suspension and distribution of the at least one encapsulate throughout the product base.

### CROSS REFERENCE TO RELATED APPLICATIONS

This application claims priority to U.S. provisional patent application Ser. No. 60/752,345, filed Dec. 21, 2005, the contents of which are incorporated herein by reference.

### BACKGROUND OF THE INVENTION

Liquid personal care compositions, including soaps and body wash products, containing encapsulates, or suspended beads, are known in the art. These compositions containing suspended beads or encapsulates typically have a gel-like rheology and are suspended in solution using a gelling or other suspending agent. These agents are used to provide uniform distribution of the suspending beads or encapsulates within the product base. In addition to the gelling or suspending agent, viscosity adjustments may also be made to the liquid formulation to establish uniform distribution. In non-liquid products, which are substantially solid at room temperature and liquid at higher temperatures, the product must remain molten and thin in order to process and fill the product casts or molds, so the addition of a gelling agent to suspend the beads or encapsulates or adjustment of the viscosity is not feasible. When the beads or encapsulates are added to a molten product that does not contain a gelling or suspending agent, the beads will aggregate in the top or the bottom of the solution, depending upon the specific gravity.

Accordingly, there is a need for a personal care composition comprising a bead or encapsulate that can be used in a molten product formulation wherein the specific gravity is such that the bead or encapsulate is suspended in the product base and evenly distributed throughout the formulation.

### SUMMARY OF THE INVENTION

The present invention is a solid soap composition comprising a soap product base and encapsulates that are suspended in the soap product base, wherein the encapsulates have substantially the same density as that of the soap product base. In one embodiment, the encapsulates contain a pigment, for example, a mineral-derived pigment, that increases the density of the encapsulate. By adjusting the ratio of the density of the encapsulate to match the density of the soap product base, the encapsulates can be distributed throughout the product base.

The invention thus provides, in one embodiment, a soap bar of Formula 1 comprising:

(i) a soap product base; and

(ii) at least one encapsulate comprising an internal phase having a density-modifying agent in a hydrophobic phase, the internal phase being surrounded by an outer phase;

wherein (i) the ratio of the density of the at least one encapsulate to the density of the soap base is from 97:100 to 103:100, preferably 99:100 to 101:100; and (ii) the at least one encapsulate is distributed in the soap product base.

The soap product base is preferably a transparent or translucent composition. For example, in addition to soap (for example, sodium and/or potassium salts of medium and long chain fatty acids, for example, stearic, myristic and/or coconut acids), it may comprise: additional surfactants (for example, anionic, cationic, non-ionic, or zwitterionic surfactants, for example, sulfate and/or sulfonate surfactants, for example, sodium lauryl sulfate, sodium laureth sulfate, disodium lauryl sulfosuccinate, as well as cocamidopropyl betaine;), as well as water and humectants, for example, polyhydric alcohols (for example, glycerol, propylene glycol, dipropylene glycol, sugar alcohols and sugars). See for example, U.S. Pat. No. 6,838,420, incorporated herein by reference.

The encapsulates have an average diameter of, for example, about 100 to about 2000 microns, for example, about 600 to about 1000 microns. In one embodiment, the outer phase of the encapsulate is gelatin gum, for example, a mixture of fish gelatin and gum Arabic, and the inner phase comprises a semi-solid oil, preferably a substance providing a skin benefit, for example, shea butter, cocoa butter or petrolatum. The semi-solid oil can be mixed with the pigment (for example, titanium dioxide where white is desired, iron oxide where red is desired, ground ultramarine for blue, and so forth) prior to encapsulation to achieve the desired density. The encapsulate may optionally comprise other materials, for example, fragrances, vitamins, etc., so long as the final mix is such that the density of the encapsulate matches that of the base during the manufacturing process.

### DETAILED DESCRIPTION OF THE INVENTION

The present invention is directed to a soap composition comprising a soap product base and encapsulates dispersed within the product base. Adjustments are made to the specific gravity of the composition such that the encapsulates are dispersed throughout the product base.

The compositions as provided herein are described and claimed with reference to their ingredients, as is usual in the art. As would be evident to one skilled in the art, the ingredients may in some instances react with one another, so that the true composition of the final formulation may not correspond exactly to the ingredients listed. Thus, it should be understood that the invention extends to the product of the combination of the listed ingredients.

All ingredients for use herein are preferably cosmetically acceptable. By “cosmetically acceptable” is meant suitable for use in a formulation for topical application to human skin. A cosmetically acceptable excipient, for example, is an excipient which is suitable for external application in the amounts and concentrations contemplated in the formulations of this invention, and includes for example excipients which are “Generally Recognized as Safe” (GRAS) by the United States Food and Drug Administration. Heavy metals, radioactive elements, and other materials which one skilled in the art would reasonably expect to be toxic for topical administration in the amounts contemplated are preferably excluded.

The soap composition of the present invention comprises a product base and one or more encapsulates in the product base. The soap product base is a soap-based composition comprising soap, surfactants, and water, and may include additional elements such as a humectant, an alcohol or desired aesthetic additives such as a fragrance or a moisturizer.

The encapsulates of the present invention have a hydrophobic internal phase. The internal phase contains a density-modifying agent and a carrier agent to provide for easier distribution of the density-modifying agent. Suitable density-modifying agents include metal oxides and other pigments made from mineral powders, as they are relatively heavy. Titanium dioxide is a pigment used in the preferred embodiment as a density-modifying agent. Other density-modifying agents may be used in the composition, if such agents impart the desired density of the encapsulate and the specific gravity of the composition. Through variance of the amount of the density-modifying agent in the encapsulate, the specific gravity is adjusted such that the encapsulate can be uniformly distributed in the product base.

The product base of the present invention preferably comprises a soap, surfactant, structurant and water.

In one embodiment, the soap product base is comprised of salts of fatty acids selected from stearic acid, myristic acid and coconut acid. Examples of additional surfactants that may be present in the soap composition include anionic, amphoteric, non-ionic and cationic surfactants. The amount of the soap is about 3 to about 40% by weight, or about 10 to about 30%. The additional surfactant is present in the soap composition in an amount by weight of about 4 to about 40%.

A structurant may be used in the product base as a substance to make the soap composition harder. Structurants such as alkali halides and alkali metal sulfates are preferred, including sodium chloride and sodium sulfate. Levels of structurant are can be 0 to about 5% by weight, or about 0.5 to about 1.5%.

Optionally, a gellant may be used in the product base to increase the bar hardness.

A humectant may be added to the product base to assist in solublizing the soap. Such humectants include polyhydric alcohols such as propylene glycol, dipropylene glycol, glycerin, sorbitol, mannitol, xylitol and hexylene glycol.

Optional ingredients which can be present in the product base include fragrances, dyes, chelating agents such as EDTA, antimicrobial agents, moisturizers and preservatives.

The bars can be prepared by standard procedures. The bars are prepared by pressing (molding) or pouring (cast) methodologies, i.e., placing a liquid into a mold.

The examples and other statements of preferred ingredients, formulations and utilities are intended to illustrate rather than to limit the invention.

The percentages described in the examples below are in weight percents based on the total formulation of that specific component of the soap composition as 100%. For example, in the Product Base example, the Soap is present in an amount by weight of about 10 to about 30% of 100% of the Product Base composition, not of the total soap composition.

The invention thus provides in a particular embodiment, a soap bar according to Formula 1 above, as follows:

Soap Product Base comprising

| Component | Wt % |
|---|---|
| Soap | 10-35 |
| Other Surfactants | 10-25 |
| Humectants | 15-30 |
| Water | QS |

Internal Phase comprising

| Component | Wt % |
|---|---|
| Shea Butter | 90-95 |
| Titanium Dioxide | 5-10 |

Outer Phase comprising

| Component | Wt % |
|---|---|
| Fish Gelatin | 40-60 |
| Gum Arabic | 40-60 |

Soap Bar Composition

| Component | Wt % |
|---|---|
| Product Base | 99.2-99.8% |
| Encapsulates | 0.2-0.8 |

Examples of representative formulations are as follows:

| Example 1 | Wt. % | Example 2 | Wt. % |
|---|---|---|---|
| Propylene Glycol | 12 | Propylene Glycol | 16 |
| Glycerin | 5 | Glycerin | 4 |
| Sorbitol | 7 | Dipropylene Glycol | 2 |
| Sodium Laureth | 12 | Sodium Laureth Sulfate | 12 |
| Sulfate | | | |
| Cocoamido Propyl | 2 | Cocomonoethanol amide 2EO | 5 |
| Betaine | | | |
| Disodium Lauryl | 8 | Cocoamido Propyl Betaine | 8 |
| Sulfosuccinate | | | |
| Sodium Chloride | 1.5 | Sodium Chloride | 1 |
| Stearic Acid | 9.5 | Stearic Acid | 14 |
| Myristic Acid | 4.2 | Myristic Acid | 7.3 |
| Coconut Acid | 3.2 | Coconut Acid | 3.5 |
| Sodium Hydroxide | 5.5 | Sodium Hydroxide | 7.44 |
| Sucrose | 2.0 | Sucrose | 4.0 |
| Triethanolamine | 1.5 | Ethanol | 1.5 |
| Ethanol | 0.5 | Fragrance | 1.0 |
| Fragrance | 1.0 | Encap (shea butter/TiO2) | 0.4 |
| Encap (shea butter/ | 0.4 | Water | q.s. |
| TiO2) | | | |
| Water | q.s. | | |

We claim:

1. A soap bar comprising:
(a) a soap product base; and
(b) at least one encapsulate comprising an internal phase having a density-modifying agent in a hydrophobic phase, the internal phase being surrounded by an outer phase;
wherein (i) the ratio of the density of the at least one encapsulate to the density of the soap base is from 97:100 to 103:100 (ii) the at least one encapsulate is distributed in the soap product base.

2. The soap bar of claim 1, wherein the at least one encapsulate independently has an average diameter of about 100 to about 2000 microns.

3. The soap bar of claim 1, wherein the outer phase of the at least one encapsulate comprises gelatin, and the inner phase comprises a semi-solid oil.

4. The soap bar of claim 3, wherein the semi-solid oil comprises an agent selected from the group consisting of shea butter, cocoa butter, and petrolatum.

5. The soap bar of claim 1, wherein the at least one encapsulate comprises a density modifying agent.

6. The soap bar of claim 1, wherein the density-modifying agent is a mineral-based pigment.

7. The soap bar of claim 1, wherein the density-modifying agent comprises 5-10% by weight of the internal phase.

8. The soap bar of claim 1, wherein the soap product base is transparent or translucent.

9. The soap bar of claim 4, wherein the semi-solid oil comprises shea butter.

10. The soap bar of claim 1 wherein the soap product base comprises:
a) a sodium salt of a fatty acid selected from the group consisting of stearic acid, myristic acid and coconut acid, in an amount by weight of about 10 to about 30%;
b) additional surfactants, in an amount by weight of about 10 to about 25%;
c) polyhydric alcohols, in an amount by weight of about 15 to about 30%.

11. The soap bar of claim 10, wherein the polyhydric alcohols comprise one or more polyhydric alcohols selected from the group consisting of glycerin, propylene glycol, dipropylene glycol, sorbital, mannitol, xylitol, hexylene glycol, sugars and mixtures thereof.

12. The soap bar of claim 11, wherein the polyhydric alcohols are selected from one or more of the group consisting of glycerin, propylene glycol, dipropylene glycol, sorbital, sucrose and mixtures thereof.

13. The soap bar of claim 10, wherein the additional surfactants comprise one or more surfactants selected from the group consisting of sodium laureth sulfate, cocamidopropylbetaine, disodium lauryl sulfosuccinate, and mixtures thereof.

14. The soap bar of claim 10, wherein the soap product base further comprises a structurant component in an amount of up to about 5% by weight.

15. The soap bar of claim 13, wherein the structurant component comprises sodium chloride.
